(19) United States
(12) Patent Application Publication
(10) Pub. No.: US 2004/0033970 A1
(43) Pub. Date: Feb. 19, 2004

(54) Antibacterial compounds with improved pharmacokinetic profiles

(76) Inventors: Richard F. Clark, Gurnee, IL (US); Stevan Djuric, Libertyville, IL (US); Zhenkun Ma, Dallas, TX (US); Ly Phan, Malden, MA (US); Michael Rupp, Indianapolis, IN (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/422,111

(22) Filed: Apr. 24, 2003

Related U.S. Application Data
(60) Provisional application No. 60/377,001, filed on Apr. 30, 2002.

(51) Int. Cl. A61K 31/7052; C07H 17/08

(52) U.S. Cl. 514/29; 536/7.4

(57) ABSTRACT

Antibacterial compounds with improved pharmacokinetic profiles having formula (I)

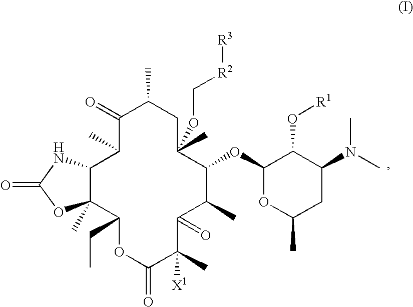

and salts, prodrugs, and salts of prodrugs thereof, processes for making the compounds and intermediates used in the processes, compositions containing the compounds, and methods for prophylaxis and treatment of bacterial infections using the compounds are disclosed.

This application claims benefit of co-pending U.S. Provisional Application Serial No. 60/377,001, filed Apr. 30, 2002, the specification of which is hereby incorporated into this application by reference.

### TECHNICAL FIELD

This invention is directed to compounds which are useful as antibacterials with improved pharmacokinetic profiles, processes for making the compounds and intermediates used in the processes, compositions containing the compounds, and methods for prophylaxis and treatment of bacterial infections using the compounds.

### BACKGROUND OF THE INVENTION

Because the effectiveness of many drugs currently available for prophylaxis and treatment of bacterial infections is being compromised by the emergence of drug-resistant bacteria, novel antibacterial compounds with improved pharmacokinetic profiles would be beneficial for their therapeutic value and their contribution to the antibacterial arts.

### SUMMARY OF THE INVENTION

A first embodiment of this invention, therefore, is directed to compounds which are useful as antibacterials with improved pharmacokinetic profiles, and salts, prodrugs, and salts of prodrugs thereof, the subset of compounds having formula (I)

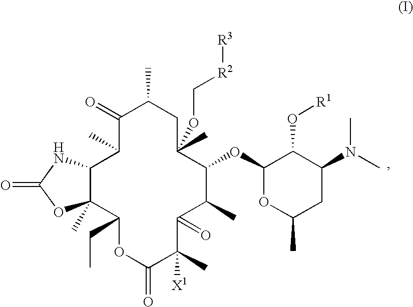

in which

R¹ is hydrogen or R^{p}, in which R^{p} is acetyl, benzoyl, trimethylsilyl, or triethylsilyl;

R² is —CH═CH— or —C≡C—;

R³ is tetraazolyl or R⁴;

R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl, thienyl, 1,3,5-triazinyl, or 1,2,3-triazolyl; and

X¹ is hydrogen or fluoro;

in which each R⁴ moiety is connected through a carbon atom, substituted with one tetraazolyl substituent, and further unsubstituted or substituted with one or two substituents independently selected from the group consisting of alkyl, alkenyl, alkynyl, halo, —CN, —OH, —NH₂, —NH(alkyl), —N(alkyl)₂, —NO₂, —CF₃, —CH₂CF₃, —CF₂CF₃, —OCF₃, —OCH₂CF₃, —OCF₂CF₃, —C(O)H, —C(O)(alkyl), —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂, and

in which the R³ tetraazolyl and the R⁴ tetraazolyl are connected through a carbon atom and are independently unsubstituted or substituted on one 1H or 2H nitrogen atom with one substituent selected from the group consisting of alkyl, —(CH₂)alkenyl, —(CH₂)alkynyl, and alkyl substituted with one substituent selected from the group consisting of halo, —CN, —OH, —NH₂, —NH(alkyl), —N(alkyl)₂, —CF₃, —CH₂CF₃, —CF₂CF₃, —C(O)H, ═O, —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂.

A second embodiment of this invention is directed to processes for making the compounds.

A third embodiment of this invention is directed to intermediates which are employed in the second embodiment.

A fourth embodiment of this invention is directed to compositions which are useful for prophylaxis or treatment of bacterial infections in a fish or a mammal, the compositions comprising a therapeutically effective amount of one or more of the compounds of the first embodiment and an excipient.

A fifth embodiment of this invention is directed to methods for prophylaxis or treatment of bacterial infections in a fish or a mammal comprising administering to the fish or the mammal a therapeutically effective amount of one or more of the compounds of the first embodiment.

### DETAILED DESCRIPTION OF THE INVENTION

Compounds of this invention, also referred to as “the compounds,” comprise both fixed and variable “moieties,” which variable moieties are identified by a capital letter and accompanying numerical or alphabetical superscript, and for which the following terms have the meanings indicated.

“Alkenyl” means monovalent, straight-chain and branched-chain hydrocarbon moieties, having two to eight carbon atoms and at least one carbon-carbon double bond, attached through a carbon atom.

Alkenyl moieties include but-1,3-dienyl, butenyl, but-2-enyl, ethenyl, 1-ethylhexen-2-yl, hex-3-enyl, 1-methylbutenyl, 2-methylbutenyl, 1-methylbut-2-enyl, 1-methylbut-1,3-dienyl, pentenyl, pent-2-enyl, pent-3-enyl, and propenyl.

“Alkyl” means monovalent, saturated, straight-chain and branched-chain hydrocarbon moieties, having one to six carbon atoms, attached through a carbon atom.

Alkyl moieties include butyl, 1,1,-dimethylethyl, 1,1-dimethylpropyl, 1,2-dimethylpropyl, ethyl, 1-ethylpropyl, 2-ethylpropyl, hexyl, methyl, 2-methylpropyl, 3-methylbutyl, 1-methylpentyl, 2-methylpent-3-yl, and pentyl.

“Alkynyl” means monovalent, straight-chain and branched-chain hydrocarbon moieties, having two to six carbon atoms and at least one carbon-carbon triple bond, attached through a carbon atom.

Alkynyl moieties include ethynyl (acetylenyl), pentynyl, pent-2-ynyl, pent-3-ynyl, pent-4-ynyl, 1-methylbut-2-ynyl, 2-methylbut-3-ynyl, hexynyl, hex-2-ynyl, hex-3-ynyl, hex-4-ynyl, 1-methyl-pent-2-ynyl, 1-methylenepent-3-ynyl, 1-methyl-pent-2,4-diynyl, and prop-2-ynyl (propargyl).

These variable moieties may combine to provide a sixth embodiment of this invention, which embodiment is directed to compounds having formula (I), and salts, prodrugs, and salts of prodrugs thereof, which are useful as antibacterials with improved pharmacokinetic profiles, in which

R¹ is hydrogen;

R² is —CH═CH— or —C≡C—;

R³ is tetraazolyl or R⁴;

R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl, thienyl, 1,3,5-triazinyl, or 1,2,3-triazolyl; and

X¹ is hydrogen or fluoro;

in which each R⁴ moiety is connected through a carbon atom, substituted with one tetraazolyl substituent, and further unsubstituted or substituted with one or two substituents independently selected from the group consisting of alkyl, alkenyl, alkynyl, halo, —CN, —OH, —NH₂, —NH(alkyl), —N(alkyl)₂, —NO₂, —CF₃, —CH₂CF₃, —CF₂CF₃, —OCF₃, —OCH₂CF₃, —OCF₂CF₃, —C(O)H, —C(O)(alkyl), —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂, and

in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —CF₃, —CH₂CF₃, —CF₂CF₃, —C(O)H, ═O, —C(O)OH, —C(O)O(alkyl), —C(O)NH₂, —C(O)NH(alkyl), —C(O)N(alkyl)₂, —OC(0) (alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂;

compounds having formula (I), and salts, prodrugs, and salts of prodrugs thereof, in which

R¹ is hydrogen;

R² is —CH═CH— or —C≡C—;

R³ is tetraazolyl or R⁴;

R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl, thienyl, 1,3,5-triazinyl, or 1,2,3-triazolyl; and

X¹ is hydrogen or fluoro;

in which each R⁴ moiety is connected through a carbon atom, substituted with one tetraazolyl substituent, and further unsubstituted or substituted with one substituent selected from the group consisting of alkyl, halo, —CN, —OH, —NH₂, —NH(alkyl), —N(alkyl)2, —NO₂, —CF₃, —C(O)H, —C(O)(alkyl), —C(O)OH, —C(O)O(alkyl), and —C(O)NH₂, and

in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —C(O)H, —C(O)OH, —C(O)O(alkyl), —C(O)NH₂, and —C(O)NH(alkyl);

compounds having formula (I), and salts, prodrugs, and salts of prodrugs thereof, in which

R¹ is hydrogen;

R² is —CH═CH— or —C≡C—;

R³ is tetraazolyl or R⁴;

R⁴ is thiazolyl, pyridyl, or thienyl; and

X¹ is hydrogen or fluoro;

in which each R⁴ moiety is connected through a carbon atom, substituted with one tetraazolyl substituent, and further unsubstituted or substituted with one substituent selected from the group consisting of alkyl, halo, —CN, —OH, —NH₂, —NH(alkyl), —N(alkyl)₂, —NO₂, —CF₃, —C(O)H, —C(O)(alkyl), —C(O)OH, —C(O)O(alkyl), and —C(O)NH₂, and

in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —C(O)H, —C(O)OH, —C(O)O(alkyl), —C(O)NH₂, and —C(O)NH(alkyl);

compounds having formula (I), and salts, prodrugs, and salts of prodrugs thereof, in which

R¹ is hydrogen;

R² is —CH═CH— or —C≡C—;

R³ is tetraazolyl or R⁴;

R⁴ is thiazolyl, pyridyl, or thienyl; and

X¹ is hydrogen or fluoro;

in which each R⁴ moiety is connected through a carbon atom, substituted with one tetraazolyl substituent, and

in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —C(O)H, —C(O)OH, —C(O)O(alkyl), —C(O)NH₂, and —C(O)NH(alkyl); and

compounds having formula (I), and salts, prodrugs, and salts of prodrugs thereof, in which

R¹ is hydrogen;

R² is —CH═CH— or —C≡C—;

R³ is 1H-tetraazol-5-yl, 2H-tetraazol-5-yl, 1-methyl-1H-tetraazol-5-yl, 2-methyl-2H-tetraazol-5-yl, 2-allyl-2H-tetraazol-5-yl, 2-prop-2-ynyl-2H-tetraazol-5-yl, 2-(((methoxy)carbonyl)methyl)-2H-tetraazol-5-yl, 2-(2-(cyano)ethyl)-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and

X¹ is hydrogen or fluoro.

A specific example of R¹ moiety for the practice of this invention is hydrogen.

Specific examples of R² moiety for the practice of this invention are —CH═CH— and —C≡C—.

Specific examples of R³ moiety for the practice of this invention are 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, and 5-(2H-tetraazol-5-yl)thien-2-yl.

Specific examples of X¹ moiety for the practice of this invention are hydrogen and fluoro.

These specific moieties of the compounds may combine with the fixed moieties thereof to form a seventh embodiment of this invention, which embodiment is directed to compounds, and salts, prodrugs, and salts of prodrugs thereof, which are useful as antibacterials with improved pharmacokinetic profiles, having formula (I)

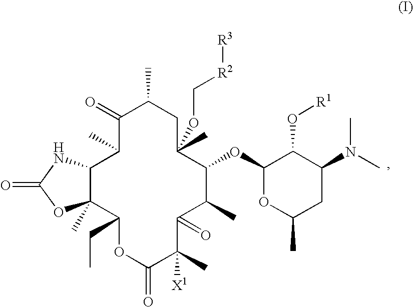

in which

R¹ is hydrogen; R² is —CH═CH— or —C≡C—; R³ is tetraazolyl or R⁴; R⁴ is thiazolyl, pyridyl, or thienyl; and X¹ is hydrogen or fluoro;

in which each R⁴ moiety is substituted with one tetraazolyl substituent, and the R³ tetraazolyl is substituted at the 1H or 2H nitrogen atom with alkyl, and the R⁴ tetraazolyl substituent is unsubstituted or substituted at the 1H or 2H nitrogen atom with one substituent selected from the group consisting of alkyl, —(CH₂)alkenyl, and alkyl substituted with one substituent selected from the group consisting of —CN and —C(O)O(alkyl);

compounds having formula (I), and salts, prodrugs, and salts of prodrugs thereof,

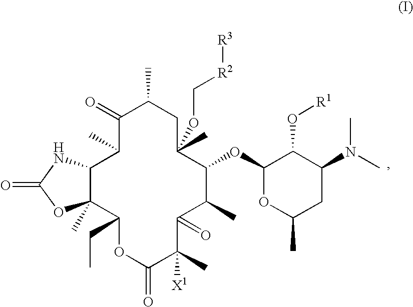

in which

R¹ is hydrogen; R² is —CH═CH— or —C≡C—; R³ is tetraazolyl or R⁴; R⁴ is thiazolyl, pyridyl, or thienyl; and X¹ is hydrogen or fluoro;

in which each R⁴ moiety is substituted with one tetraazolyl substituent, and the R³ tetraazolyl is substituted at the 1H or 2H nitrogen atom with C₁-alkyl, and the R⁴ tetraazolyl substituent is unsubstituted or substituted at the 1H or 2H nitrogen atom with one substituent selected from the group consisting of C₁-alkyl, —(CH₂)—C₂-alkenyl, and C₁-C₂-alkyl substituted with one substituent selected from the group consisting of —CN and —C(O)O(C₁-alkyl);

compounds having formula (I), and salts, prodrugs, and salts of prodrugs thereof, in which R¹ is hydrogen; R² is —CH═CH—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and X¹ is hydrogen;

compounds having formula (I), and salts, prodrugs, and salts of prodrugs thereof, in which R¹ is hydrogen; R² is —CH═CH—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and X¹ is fluoro;

compounds having formula (I), and salts, prodrugs, and salts of prodrugs thereof, in which R¹ is hydrogen; R² is —C≡C—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(l-methyl-1H-tetraazol-5-yl)thien-2-yl, or 5-(2H-tetraazol-5-yl)thien-2-yl; and X¹ is hydrogen; and

compounds having formula (I), and salts, prodrugs, and salts of prodrugs thereof, in which R¹ is hydrogen; R² is —C≡C—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and X is fluoride; and

an eighth embodiment of this invention, which embodiment is directed to compounds, and salts, prodrugs, and salts of prodrugs thereof, which compounds include

(3aS,4R,7S,9R,10R,11S,13R,15R,15aR)-4-ethyl-7-fluoro-3a,7,9,11,13,15-hexamethyl-1-((3-(2-(2-methyl-2H-tetraazol-5-yl)-1,3-thiazol-5-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

(3aS,4R,7R,9R,10R,11S,13R,15R,15aR)-4-ethyl-3a,7,9,11,13,15-hexamethyl-1-((3-(2-(2-methyl-2H-tetraazol-5-yl)-1,3-thiazol-5-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

(3aS,4R,7R,9R,10R,11S,13R,15R,15aR)-4-ethyl-3a,7,9,11,13,15-hexamethyl-11-(((2E)-3-(5-(2-methyl-2H-tetraazol-5-yl)thien-2-yl)prop-2-enyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

(3aS,4R,7R,9R,10R,11S,13R,15R,15aR)-4-ethyl-3a,7,9,11,13,15-hexamethyl-11-(((2E)-3-(5-(1-methyl-1H-tetraazol-5-yl)thien-2-yl)prop-2-enyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

(3aS,4R,7R,9R,10R,11S,13R,15R,15aR)-4-ethyl-3a,7,9,11,13,15-hexamethyl-2,6,8,14-tetraoxo-11-(((2E)-3-(5-(2H-tetraazol-5-yl)thien-2-yl)prop-2-enyl)oxy)-tetradecahydro-2H-oxacyclotetradecino(4,3-d)(1,3)oxazol-10-yl 3,4,6-trideoxy-3-(dimethylamino)-β-D-xylo-hexopyranoside;

(3aS,4R,7S,9R,10R,11S,13R,15R,15aR)-4-ethyl-7-fluoro-3a,7,9,11,13,15-hexamethyl-11-((3-(5-(2-methyl-2H-tetraazol-5-yl)thien-2-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

(3aS,4R,7R,9R,10R,11S,13R,15R,15aR)-4-ethyl-3a,7,9,11,13,15-hexamethyl-11-((3-(5-(2-methyl-2H-tetraazol-5-yl)thien-2-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

(3aS,4R-,7S,9R,10R,11S,13R,15R,15aR)-4-ethyl-7-fluoro-3a,7,9,11,13,15-hexamethyl-11-(((2E)-3-(5-(2-methyl-2H-tetraazol-5-yl)thien-2-yl)prop-2-enyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

methyl (5-(5-(3-(((3aS,4R,7S,9R,10R,11S,13R,15R,15aR)-4-ethyl-7-fluoro-3a,7,9,11,13,15-hexamethyl-2,6,8,14-tetraoxo-10-((3,4,6-trideoxy-3-(dimethylamino)-beta-D-xylo-hexopyranosyl)oxy)tetradecahydro-2H-oxacyclotetradecino (4,3-d)(1,3)oxazol-11-yl)oxy)prop-1-ynyl)thien-2-yl)-2H-tetraazol-2-yl)acetate;

(3aS,4R,7S,9R,10R,11S,13R,15R,15aR)-4-ethyl-7-fluoro-3a,7,9,11,13,15-hexamethyl-1-((3-(6-(2-methyl-2H-tetraazol-5-yl)pyridin-3-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

(3aS,4R,7S,9R,10R,11S,13R,15R,15aR)-11-((3-(5-(2-allyl-2H-tetraazol-5-yl)thien-2-yl)prop-2-ynyl)oxy)-4-ethyl-7-fluoro-3a,7,9,11,13,15-hexamethyl-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;

3-(5-(5-(3-(((3aS,4R,7S,9R,10R,11S,13R,15R,15aR)-4-ethyl-7-fluoro-3a,7,9,11,13,15-hexamethyl-2,6,8,14-tetraoxo-10-((3,4,6-trideoxy-3-(dimethylamino)-β-D-xylo-hexopyranosyl)oxy)tetradecahydro-2H-oxacyclotetradecino-(4,3-d)(1,3)oxazol-11-yl)oxy)prop-1-ynyl)thien-2-yl)-2H-tetraazol-2-yl)propanenitrile; and

(3aS,4R,7S,9R,10R,11S,13R,15R,15aR)-4-ethyl-7-fluoro-3a,7,9,11,13,15-hexamethyl-11-((3-(2-methyl-2H-tetraazol-5-yl)prop-2-ynyl)oxy)-2,6,8,14-tetraoxotetradecahydro-2H-oxacyclotetradecino(4,3-d)(1,3)oxazol-10-yl 3,4,6-trideoxy-3-(dimethylamino)-β-D-xylo-hexopyranoside.

Compounds of this invention contain asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. Compounds having asymmetrically substituted carbon atoms with equal amounts of R and S configurations are racemic at those carbon atoms. Atoms with an excess of one configuration over the other are assigned the configuration which is present in the higher amount, preferably an excess of about 85%-90%, more preferably an excess of about 95%-99%, and still more preferably an excess greater than about 99%. Accordingly, this invention is meant to embrace racemic mixtures, relative and absolute stereoisomers, and mixtures of relative and absolute stereoisomers of the compounds.

Compounds of this invention may also contain carbon-carbon double bonds or carbon-nitrogen double bonds in the Z or E configuration, in which the term “Z” represents the larger two substituents on the same side of a carbon-carbon or carbon-nitrogen double bond and the term “E” represents the larger two substituents on opposite sides of a carbon-carbon or carbon-nitrogen double bond. The compounds may also exist as an equilibrium mixture of Z or E configurations.

Compounds of this invention which contain —OH, —NH—, or —CO₂H moieties may have attached thereto prodrug-forming moieties. The prodrug-forming moieties are removed by metabolic processes and release the compounds having the freed hydroxyl, amino, or carboxylic acid in vivo. Prodrugs are useful for adjusting such pharmacokinetic properties of the compounds as solubility and/or hydrophobicity, absorption in the gastrointestinal tract, bioavailability, tissue penetration, and rate of clearance.

Compounds of this invention may exist as acid addition salts, basic addition salts, or zwitterions. Salts of the compounds are prepared during their isolation or following their purification. Acid addition salts of the compounds are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate, salts of the compounds and prodrugs thereof are contemplated as being embraced by this invention. When the compounds contain carboxylic acids, basic addition salts may be prepared therefrom by reaction with a base such as the hydroxide, carbonate, and bicarbonate, of cations such as lithium, sodium, potassium, and calcium, magnesium.

Compounds of this invention may be administered with or without an excipient. Excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. Excipients for orally administered compounds in solid dosage forms include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodiumphosphate salts, soybean oil, sucrose, tetrahydrofurfuryl alcohol, and mixtures thereof. Excipients for ophthalmically and orally administered compounds in liquid dosage forms include benzyl alcohol, benzyl benzoate, 1,3-butylene glycol, castor oil, corn oil, cottonseed oil, ethanol, ethyl acetate, ethyl carbonate, fatty acid esters of sorbitan, germ oil, groundnut oil, glycerol, isopropanol, olive oil, polyethylene glycols, propylene glycol, sesame oil, tetrahydrofurfuryl alcohol, water, and mixtures thereof. Excipients for osmotically administered compounds include chlorofluorohydrocarbons, ethanol, isopropanol, water, and mixtures thereof. Excipients for parenterally administered compounds include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for rectally and vaginally administered compounds include cocoa butter, polyethylene glycol, wax, and mixtures thereof.

Compounds of this invention may be administered orally, ophthalmically, osmotically, parenterally (subcutaneously, intramuscularly, intrasternally, intravenously), rectally, topically, transdermally, and vaginally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous suspensions, which suspensions comprise crystalline, amorphous, or otherwise insoluble forms of the compounds. Rectally and vaginally administered compounds may be administered as creams, gels, lotions, ointments, and pastes.

Therapeutically effective amounts of compounds of this invention depend on the recepient of treatment, the disorder being treated and the severity of the disorder, the composition comprising the compounds, the time of administration, the route of administration, the duration of treatment, the potency of the compounds, and the rate of excretion of the compounds. The daily therapeutically effective amount of the compounds administered to a patient in single or divided doses range from about 0.1 to about 200 mg/kg body weight, preferably from about 0.25 to about 100 mg/kg body weight. Single dose compositions contain these amounts of the compounds or combinations of submultiples thereof.

To determine antibacterial activity of compounds of this invention, twelve petri dishes, each containing successive aqueous dilutions of test compounds in sterilized Brain Heart Infusion agar (Difco 0418-01-5) (10 mL), were inoculated with 1:100 dilutions of the representative microorganisms in TABLE 1 using a Steers replicator block (or 1:10 dilutions for slow-growing Streptococcus strains), co-incubated at 35-37° C. for 20-24 hours with a control plate having no compound, and inspected visually to provide the minimum inhibitory concentration (MIC), in μg/mL, by which is meant the lowest concentration of the test compound which yielded no growth, a slight haze, or sparsely isolated colonies on the inoculums spot as compared to growth in the control plate.

TABLE 1

| Microorganism | Code |
|---|---|
| Staphylococcus aureus NCTC10649M | AA |
| Staphylococcus aureus A5177 | BB |
| Staphylococcus aureus PIU 2043 | CC |
| Staphylococcus aureus 1775 | DD |
| Streptococcus pyrogenes EES61 | EE |
| Streptococcus pyrogenes 930 | FF |
| Streptococcus pyrogenes PIU 2548 | GG |
| Streptococcus pneumoniae ATCC 6303 | HH |
| Streptococcus pneumoniae 5979 | JJ |
| Streptococcus pneumoniae 5649 | KK |
| Enterococcus faecalis PIU 1967 | LL |
| Enterococcus faecium GYR 1632 | MM |
| Moraxella catarrhalis 2604 | NN |
| Haemophilus influenzae GYR 1435 | PP |
| Escherichia coli JUHL | QQ |

Compounds of this invention displayed antibacterial activity in the range of about 0.005 μg/mL to greater than about 100 μg/mL against the microorganisms listed in Table 1 while the control demonstrated no antibacterial activity against these microorganisms. This antibacterial activity demonstrates the usefulness of the compounds as antibacterials.

It is meant to be understood that certain metabolites of compounds of this invention, produced by in vitro or in vivo metabolic processes, would also be useful as antibacterials and are meant to be embraced by this invention.

It is also meant to be understood that certain precursor compounds, which precursor compounds may be metabolized in vitro or in vivo to form compounds of this invention, are meant to be embraced by this invention.

The pharmacokinetic profiles of EXAMPLE 20, EXAMPLE 22, EXAMPLE 29 and EXAMPLE 31 were evaluated using cassette dosing protocols in dog. The cassette dosing protocol in dogs combined three test compounds with the reference compound (EXAMPLE 104 of commonly-owned U.S. Pat. No. 5,866,549), each at a dose of 1 mg/kg. The compounds for each cassette were formulated as a solution in an aqueous vehicle containing 10% ethanol and one equivalent hydrochloric acid. Groups of three male beagle dogs received either an intravenous dose (IV) or an oral dose (PO). Sequential blood samples were taken from each animal for 24 hours after dosing. The plasma concentrations of the test compound and reference compounds were simultaneously determined using HPLC-MS/MS following liquid-liquid extraction of the samples. The results are shown in TABLE 2, in which CL_{P} is plasma clearance; T_{1/2} and Tₘₐₓ are expressed in hours; Cₘₐₓ is expressed in μg/mL; AUC is expressed in μg·h/mL; F is percent bioavaibility; and Vc and Vb (volumes of distibution) are expressed in L/Kg.

TABLE 2

| | | T_{1/2} | Vc | Vb | AUC | CLp | T_{1/2} | Cₘₐₓ | Tₘₐₓ | AUC | F |
|---|---|---|---|---|---|---|---|---|---|---|---|
| EXAMPLE | Dose | (IV) | (IV) | (IV) | (IV) | (IV) | (PO) | (PO) | (PO) | (PO) | (PO) |
| standard | 1 | 4.7 | 1.8 | 2.4 | 3.01 | 0.35 | 4.2 | 0.21 | 1.1 | 1.79 | 59.00 |
| 20 | 1 | 14.4 | 1.6 | 3.1 | 7.29 | 0.16 | 11.1 | 0.25 | 2 | 5.14 | 73.60 |
| 22 | 1 | 8.4 | 0.9 | 1.1 | 11.52 | 0.09 | 7.8 | 0.49 | 1.5 | 6.77 | 63.00 |
| 29 | 1 | 9 | 0.9 | 1.2 | 11.9 | 0.09 | 6.7 | 0.72 | 1.3 | 9.66 | 80.30 |
| 31 | 1 | 22.9 | 1.1 | 2.3 | 16.13 | 0.07 | 17.2 | 0.48 | 4.5 | 12.6 | 81.10 |

The data in TABLE 2 show the surprising pharmacokinetic profiles of the compounds characterized by low plasma clearance values, long half lives, and high oral bioavailabilities.

Compounds of this invention may be prepared by synthetic chemical processes, examples of which synthetic chemical processes, and intermediates used in the processes, are shown hereinbelow. It is meant to be understood that the order of the steps in the processes may be varied, that like reagents, solvents, and reaction conditions may be substituted for those specifically mentioned, and that vulnerable moieties may be protected and deprotected during the process.

Abbreviations used herein are THF for tetrahydrofuran, DME for 1,2-dimethoxyethane; and DMF for N,N-dimethylformamide.

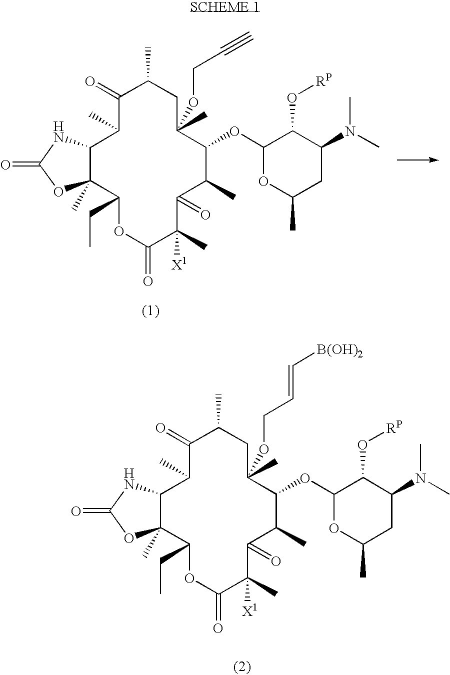

The compound having formula (1), in which X¹ is hydrogen, may be prepared as described in commonly-owned U.S. Pat. No. 5,866,549.

The compound having formula (1), in which X¹ is fluoro, may be prepared as described in commonly-owned U.S. Pat. No. 6,124,269.

Compounds having formula (1) may be converted to compounds having formula (2) by (a) reacting the former and a borane, with or without 2-methyl-2-butene; and (b) reacting the product of step (a) and water. Boranes include borane.tetrahydrofuran, borane.dimethylsulfide, and borane.1,4-oxathiane. Step (a) is typically conducted from about −5° C. to about 5° C., for about 1 to about 10 hours, in solvents such as diethyl ether, dioxane, DME, and THF. Step (b) is typically conducted, without isolation of the product of Step (a), from about −5° C. to about 5° C., for about 1 to about 10 hours, in mixtures comprising water and one or more of benzene, diethyl ether, dioxane, DME, THF, and toluene.

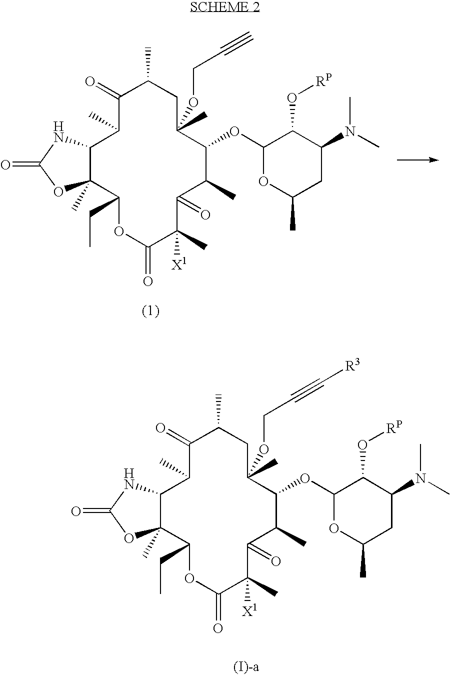

Compounds having formula (1) may be converted to compounds having formula (I)-a by reacting the former, compounds having formula (3),

X²—R³ (3),

a first base, a coupling catalyst, and copper(I) iodide, with or without an additive. First bases include N,N-diisopropylethylamine, and triethylamine. Coupling catalysts include dichlorobis(triphenylphosphine)palladium(II), tris(dibenzylideneacetone)dipalladium(0), tetrakis(triphenylphosphine)palladium(0), and dichlorobis(triphenylphosphine)nickel(II). Additives include 1,2-bis(diphenylphosphino)ethane, 1,3-bis(diphenylphosphino)propane, triphenylphosphine, triphenylarsine, and mixtures thereof. The reaction is typically conducted from about 50° C. to about 80° C., for about 12 to about 48 hours, in solvents such as acetonitrile, benzene, diethyl ether, DME, dioxane, THF, and toluene.

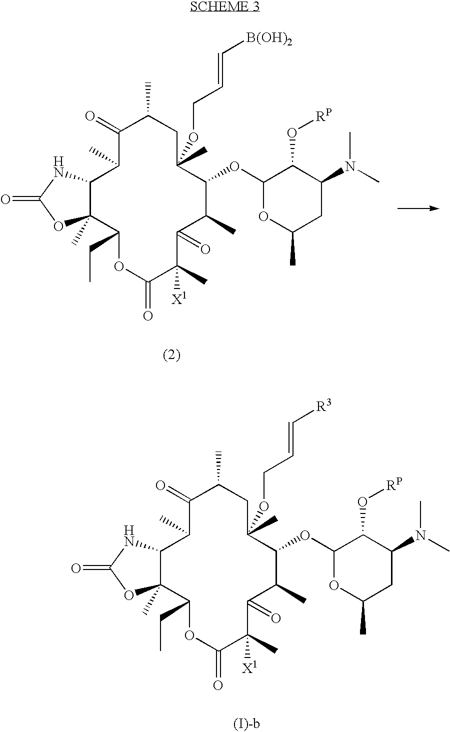

Compounds having formula (2) may be converted to compounds having formula (I)-b by reacting the former, the compounds having formula (3), the coupling catalyst, and a second base, with or without the additive.

Second bases include sodium carbonate, potassium carbonate, cesium carbonate, cesium fluoride, N,N-diisopropylethylamine, and triethylamine.

The reaction is typically conducted from about 50° C. to about 80° C., for about 12 to about 48 hours, in solvents such as acetonitrile, benzene, diethyl ether, DME, dioxane, THF, and toluene.

The compounds and processes of this invention will be better understood in connection with the following examples.

### EXAMPLE 1

This example was prepared according to EXAMPLE 246 in commonly-owned U.S. Pat. No. 5,866,549 and substituting 90% technical grade benzoic anhydride for acetic anhydride in Step 246d.

### EXAMPLE 2

This example was prepared according to EXAMPLE 4 in commonly-owned U.S. Pat. No. 6,124,269 and substituting EXAMPLE 1 for the “compound 14 of Scheme 4.”

### EXAMPLE 3

A solution of 1M borane.THF (119 mL) at 0° C. was treated with a solution of 2-methyl-2-butene (25.1 mL) in THF (75 mL) at 0° C., stirred for 45 minutes, treated with a solution of EXAMPLE 2 (15 g) in THF (100 mL) at 0° C., stirred for 2 hours, treated with 10% K₂CO₃, and extracted with ethyl acetete; and the extract was dried (Na₂SO₄), filtered, and concentrated. A solution of the concentrate in ethyl acetate (50 mL) was treated with hexane (500 mL) and filtered; and the filtrant was flash chromatographed on silica gel with 50-70% acetone/hexanes.

### EXAMPLE 4

This example was prepared by substituting EXAMPLE 1 for EXAMPLE 2 in EXAMPLE 3

### EXAMPLE 5

This example was prepared by substituting EXAMPLE 2 for “the compound from Step 1a” in Step 1b of EXAMPLE 1 of commonly-owned U.S. Pat. No. 6,124,269.

### EXAMPLE 6 and EXAMPLE 7

### 5-(5-bromothien-2-yl)-2-methyl-2H-tetraazole and 5-(5-bromothien-2-yl)-1-methyl-1H-tetraazole

A solution of 5-(5-bromothien-2-yl)-1H (and 2H)-tetraazole (750 mg) and diisopropylethylamine (850 μL) in acetonitrile (25 mL) and methanol (2.5 mL) at 0° C. was treated with 2M (trimethylsilyl)diazomethane in THF (2.4 mL), stirred for 3 hours at 25° C., treated with ethyl acetate, washed with water and brine, and dried (Na₂SO₄), filtered, and concentrated; and the concentrate was flash chromatographed on silica gel with 99:1 dichloromethane/methanol.

### EXAMPLE 8

A solution of 5-(5-bromothien-2-yl)-2H-tetraazole and 5-(5-bromothien-2-yl)-1H-tetraazole (100 mg), triethylamine (120 μL), and 2-(trimethylsilyl)ethoxymethylchloride (80 μL) in THF at 25° C. was stirred for 2 hours, treated with ethyl acetate, washed with water and brine, and dried (Na₂SO₄), filtered, and concentrated; and the concentrate was flash chromatographed on silica gel with dichloromethane.

### EXAMPLE 9

A solution of 5-(5-bromothien-2-yl)-1H-tetraazole and 5-(5-bromothien-2-yl)-2H-tetraazole (300 mg) in ethanol (5 mL) was treated with silver nitrate in water (1 mL), stirred for 30 minutes, treated with concentrated ammonium hydroxide (5 mL), and filtered. A slurry of the filtrant in chloroform (10 mL) was treated with allyl bromide (280 μL), stirred at 65° C. for 18 hours, and filtered; and the filtrant was flash chromatographed on silica gel with dichloromethane.

### EXAMPLE 10

A solution of 5-(5-bromothien-2-yl)-1H-tetraazole and 5-(5-bromothien-2-yl)-2H-tetraazole (500 mg), acrylonitrile (210 μL), and triethylamine (900 μL) in isopropanol (2 mL) was stirred at 110° C. in a sealed tube for 18 hours and concentrated; and the concentrate was flash chromatographed on silica gel with 99:1 dichloromethane/methanol.

### EXAMPLE 12

A mixture of sodium hydroxide (14.2 g) and 1H-tetraazole and 2H-tetraazole (25 g) in water (600 mL) at 25° C. was stirred until homogeneous, treated sequentially with dichloromethane (600 mL), dimethyl sulfate (47.2 g), and n-tetrabutylammonium bromide (5.7 g), stirred for 14 hours, separated from the water layer, and distilled at 1 atm with collection of the 143° C. fraction.

### EXAMPLE 13

A solution of EXAMPLE 12 (3.5 g) in THF (150 mL) at −78° C. was treated with 2.5 M n-butyllithium in cyclohexane (20.8 mL) over 35 minutes, stirred for 15 minutes, treated with a solution of N-iodosuccinimide (10.3 g) in THF (75 mL) over 20 minutes, stirred for 30 minutes, warmed to 0° C. and stirred for 40 minutes, warmed to 25° C. and stirred for 10 minutes, treated with diethyl ether, washed with 5% KH₂PO₄, water, and brine, and dried (Na₂SO₄), filtered, and concentrated; and the concentrate was flash chromatographed on silica gel with 1:1 dichloromethane/hexanes.

### EXAMPLE 14

A solution of EXAMPLE 13 (1.78 g) in toluene (19 mL) was treated with 2-tributylstannylthiazole (3.65 g) and tetrakis(triphenylphosphine)palladium (0) (196 mg), stirred at 95° C. for 16 hours, and concentrated; and the concentrate was flash chromatographed on silica gel with dichloromethane then 25:75 acetone/hexanes.

### EXAMPLE 15

A solution of 2.5M n-butyllithium in hexanes at −78° C. (1.8 mL) in diethyl ether (16 mL) was treated with a solution of EXAMPLE 14 (600 mg) in tetrahydrofuran (18 mL) over 1 hour, stirred for 45 minutes, treated with 1M trimethylstannyl chloride in THF (4.5 mL) in tetrahydrofuran (4 mL) over 10 minutes, stirred for 1 hour, and concentrated; and the concentrate was treated with ethyl ether (50 mL), filtered through diatomaceous earth (Celite®), and concentrated.

### EXAMPLE 16

A solution of the EXAMPLE 15 concentrate in THF (35 mL) at 25° C. was treated with iodine (1 g) in THF (15 mL) over 5 minutes, stirred for 2 hours, treated with ethyl acetate, washed with 5% Na₂CO₃, 10% Na₂S₂O₃, and brine, and dried (Na₂SO₄), filtered, and concentrated; and the concentrate was flash chromatographed on silica gel with of 1:1 hexanes/dichloromethane then dichloromethane.

### EXAMPLE 17

A solution of 5-bromo-2-cyanopyridine (1 g), ammonium chloride (4.3 g), and sodium azide (5.32 g) in DMF (50 mL) was stirred at 130° C. for 4 hours, cooled to ambient temperature, diluted with dichloromethane, washed with water and brine, and dried (Na₂SO₄), filtered and concentrated.

### EXAMPLE 18

A mixture of EXAMPLE 17 (582 mg) and diisopropylethylamine (650 mL) in acetonitrile (10 mL) and methanol (2 mL) at 0° C. was treated with trimethylsilyldiazomethane (1.93 mL) over 3 minutes, stirred for 1.5 hours, diluted with ethyl acetate, washed with 5% NaHCO₃, water, and brine, and dried (Na₂SO₄), filtered and concentrated; and the concentrate was flash column chromatographed on silica gel with 4:1 hexanes/ethyl acetate.

### EXAMPLE 19

A solution of EXAMPLE 2 (1.4 g), EXAMPLE 6 (500 mg) triethylamine (2 mL), and 1,2-bis(diphenylphosphino)ethane (73.7 mg) in acetonitrile (10 mL) at 25° C. was treated with tris(dibenzylideneacetone)dipalladium(0) (84.7 mg) and copper(I) iodide (17.6 mg), stirred for 10 minutes at 25° C. and 85° C. for 24 hours, and concentrated; and the concentrate was flash chromatographed on silica gel with 20-33% acetone/hexanes.

### EXAMPLE 20

A solution of EXAMPLE 19 (1.36 g) in methanol (500 mL) at reflux was stirred for 8 hours and concentrated; and the concentrate was flash chromatographed on silica gel with 97.0:2.0:1.0 dichloromethane/methanol concentrated ammonium hydroxide.

### EXAMPLE 21

A mixture of EXAMPLE 4 (3 g), EXAMPLE 6 (1.03 g), and sodium carbonate (1.21 g) in toluene (50 mL) and water (25 mL) was treated with tetrakis(triphenylphosphine)palladium(0) (440 mg), stirred at reflux for 16 hours, and concentrated; and the concentrate was flash chromatographed on silica gel with 1:1 acetone/hexanes.

### EXAMPLE 22

A solution of EXAMPLE 21 (1.03 g) in methanol (15 mL) was stirred at reflux for 8 hours and concentrated; and the concentrate was flash chromatographed on silica gel with 94.9:5:0.1 dichloromethane/methanol/concentrated ammonium hydroxide.

### EXAMPLE 23

A mixture of EXAMPLE 4 (670 mg), EXAMPLE 7 (230 mg), and sodium carbonate (270 mg) in toluene (12 mL) and water (6 mL) was treated with tetrakis(triphenylphosphine)palladium(0) (110 mg), stirred at reflux for 16 hours, and concentrated; and the concentrate was flash chromatographed on silica gel with 97.9:2:0.1 dichloromethane/methanol/concentrated ammonium hydroxide.

### EXAMPLE 24

This example was prepared by substituting EXAMPLE 23 for EXAMPLE 19 in EXAMPLE 20.

### EXAMPLE 25

A mixture of EXAMPLE 4 (1.4 g), EXAMPLE 8 (710 mg), and sodium carbonate (570 mg) in toluene (25 mL) and water (12 mL) was treated with tetrakis(triphenylphosphine)-palladium(0) (200 mg), stirred at reflux for 16 hours, and concentrated; and the concentrate was flash chromatographed on silica gel with 30% to 50% to 70% acetone/hexanes.

### EXAMPLE 26

A solution of EXAMPLE 25 (600 mg) in methanol (15 mL) was stirred at reflux for 6 hours and concentrated; and the concentrate was flash chromatographed on silica gel with 94.9:5:0.1 dichloromethane/methanol/concentrated ammonium hydroxide.

### EXAMPLE 27

A mixture of EXAMPLE 26 (320 mg), 4 Å molecular sieves, and 1M tetrabutylammonium fluoride in THF (1.751 mL) in THF (15 mL) was stirred at reflux for 4 hours and cooled, treated with chloroform, filtered, washed with water, and dried (Na₂SO₄), filtered, and concentrated; and the concentrate was flash chromatographed on silica gel with 94.5:5:0.5 dichloromethane/methanol/concentrated ammonium hydroxide.

### EXAMPLE 28

A solution of EXAMPLE 1 (1 g), EXAMPLE 6 (430 mg) triethylamine (1.4 mL), and 1,2-bis(diphenylphosphino)ethane (54 mg) in acetonitrile (7 mL) at 25° C. was treated with tris(dibenzylideneacetone)dipalladium(0) (62 mg) and copper(I) iodide (12.8 mg), stirred for 1 hour at 25° C. and at 85° C. for 24 hours, and concentrated; and the concentrate was flash chromatographed on silica gel with 20-33% acetone/hexanes.

### EXAMPLE 29

A solution of EXAMPLE 28 (790 mg) in methanol (15 mL) was stirred at reflux for 16 hours and concentrated; and the concentrate was flash chromatographed on silica gel with 97:2:1 dichloromethane/methanol/concentrated ammonium hydroxide.

### EXAMPLE 30

A solution of EXAMPLE 3 (600 mg), EXAMPLE 6 (365 mg), and potassium carbonate (206 mg) in acetone (5 mL) and water (5 mL) was treated with palladium(II) acetate (8.4 mg), stirred at 65° C. for 16 hours and cooled, treated with ethyl acetate, washed with water and brine, and dried (Na₂SO₄), filtered, and concentrated.

### EXAMPLE 31

A solution of EXAMPLE 30 (600 mg) in methanol (15 mL) was stirred at reflux for 16 hours and concentrated; and the concentrate was flash chromatographed on silica gel with 98:2 dichloromethane/methanol.

### EXAMPLE 33

This example was prepared by substituting EXAMPLE 28 for EXAMPLE 19 in EXAMPLE 20.

### EXAMPLE 34

This example was prepared by substituting EXAMPLE 9 for EXAMPLE 6 in EXAMPLE 19

### EXAMPLE 35

A solution of EXAMPLE 34 (50 mg) in methanol (5 mL) was stirred at reflux for 16 hours and concentrated; and the concentrate was flash chromatographed on silica gel with 97:2:1 dichloromethane/methanol/concentrated ammonium hydroxide.

### EXAMPLE 36

A solution of EXAMPLE 5 (417 mg), EXAMPLE 18 (153 mg), and triethylamine (1 mL) in acetonitrile (10 mL) at 25° C. was treated with bis(triphenylphosphine)palladium(II) acetate (44.7 mg) and copper(I) iodide (2.9 mg), stirred at 90° C. for 4 hours, and concentrated; and the concentrate was flash chromatographed on silica gel with 2% methanol/dichloromethane.

### EXAMPLE 37

A solution of EXAMPLE 5 (630 mg), EXAMPLE 10 (300 mg) triethylamine (3 mL), and 1,2-bis(diphenylphosphino)ethane (38 mg) in acetonitrile (3 mL) at 25° C. was treated with tris(dibenzylideneacetone)dipalladium(0) (40 mg) and copper(I) iodide (4 mg), stirred for 18 hours at 85° C., and concentrated; and the concentrate was flash chromatographed on silica gel with 94.5:5:0.1 dichloromethane/methanol/ concentrated ammonium hydroxide.

### EXAMPLE 38

A solution of EXAMPLE 2 (700 mg), EXAMPLE 13 (230 mg), and triethylamine (5 mL) in acetonitrile (10 mL) at 25° C. was treated with bis(triphenylphosphine)palladium(II) acetate (32 mg) and copper(I) iodide (4 mg), stirred at 90° C. for 4 hours, and concentrated; and the concentrate was flash chromatographed on silica gel with 1:1 hexane/acetone.

### EXAMPLE 39

This example was prepared by substituting EXAMPLE 38 for EXAMPLE 19 in EXAMPLE 20.

### EXAMPLE 40

A solution of EXAMPLE 2 (800 mg) and triethylamine (3 ml) in acetonitrile (16 mL) at 25° C. was treated sequentially with EXAMPLE 16 (324 mg), dichlorobis(triphenylphosphine)palladium(II) (52 mg), and copper(I) iodode (4 mg), stirred at 65° C. for 16 hours and concentrated; and the concentrate was flash chromatographed on silica gel with 25:75 acetone/hexanes.

### EXAMPLE 41

This example was prepared by substituting EXAMPLE 40 for EXAMPLE 19 in EXAMPLE 20.

### EXAMPLE 42

A solution of EXAMPLE 1 (220 mg) and triethylamine (750 μl) in acetonitrile (4 mL) at 25° C. was treated sequentially with EXAMPLE 16 (100 mg), dichlorobis(triphenylphosphine)palladium(II) (15 mg), and copper(I) iodode (1 mg), stirred at 65° C. for 16 hours and concentrated; and the concentrate was flash chromatographed on silica gel with 25:75 acetone/hexanes.

### EXAMPLE 43

A solution of EXAMPLE 42 (190 mg) in methanol (8 mL) was stirred reflux for 16 hours and concentrated; and the concentrate was flash chromatographed on silica gel with 99:1 dichloromethane/methanol then 98.5:1:0.5 dichloromethane/methanol/concentrated ammonium hydroxide.

### EXAMPLE 20

¹³C NMR (75 MHz, CDCl₃) δ 216.6, 204.0, 203.8, 165.9, 165.7 (C-1), 160.7, 157.2, 133.0, 129.9, 127.5, 125.3, 104.1, 91.9, 83.4, 80.2, 80.0, 78.7, 78.5, 70.3, 69.7, 65.8, 58.1, 51.1, 44.1, 40.5, 40.2, 39.4, 38.4, 37.4, 28.1, 25.3, 25.1, 22.2, 21.1, 20.2, 17.6, 15.3, 13.7, 13.2, 10.6.

### EXAMPLE 22

¹³C NMR (75 MHz, CDCl₃) δ 217.1, 205.23, 169.6, 161.2, 157.5, 144.0, 128.1, 126.9, 126.6, 126.1, 102.9, 83.4, 78.6, 77.6, 76.4, 70.2, 69.5, 65.9, 63.8, 58.2, 50.9, 46.2, 45.0, 40.2, 39.3, 38.9, 37.3, 28.3, 22.7, 21.2, 20.3, 18.1, 14.4, 14.1, 13.7, 10.7.

### EXAMPLE 24

¹³C NMR (75 MHz, CDCl₃) δ 217.4, 205.1, 169.5, 157.5, 149.8, 146.5, 130.76, 128.4, 126.7, 125.63, 122.4, 102.9, 83.52, 78.63, 77.4, 76.3, 70.1, 69.5, 65.8, 63.5, 58.1, 50.8, 46.3, 44.9, 40.2, 38.8, 37.2, 35.1, 28.2, 22.6, 21.2, 20.4, 17.9, 14.34, 14.31, 13.58, 13.55, 10.7.

### EXAMPLE 27

¹³C NMR (75 MHz, CDCl₃) δ 217.2, 205.2, 169.6, 157.6, 156.7, 141.5, 126.5, 126.3, 126.1, 125.7, 102.6, 83.6, 78.6, 77.2, 70.1, 69.1, 65.9, 63.9, 58.6, 52.2, 50.8, 45.0, 40.2, 38.8, 37.3, 30.9, 25.5, 23.7, 22.6, 21.1, 20.2, 19.6, 18.1, 14.5, 14.2, 13.6, 10.6.

### EXAMPLE 29

¹³C NMR (75 MHz, CDCl₃) δ 216.9, 205.2, 169.4, 157.7, 133.2, 130.0, 127.5, 125.4, 103.1, 91.4, 83.6, 79.5, 78.7, 77.4, 77.2, 70.3, 69.6, 65.9, 58.2, 51.7, 51.1, 46.7, 44.8, 40.2, 39.5, 38.8, 37.4, 28.2, 22.5, 21.2, 19.7, 18.0, 14.7, 14.5, 13.6, 13.5, 10.6.

### EXAMPLE 31

¹³C NMR (CDCl₃, 75 MHz) δ 217.6, 204.0, 203.6, 166.0, 165.6, 161.3, 157.0, 143.9, 128.3, 127.8, 127.7, 126.7, 126.2, 103.9, 99.2, 83.3, 79.9, 79.2, 79.0, 70.3, 69.6, 65.9, 63.7, 58.2, 44.2, 40.6, 40.2, 39.4, 38.8, 37.5, 28.4, 25.5, 25.3, 25.2, 22.4, 21.1, 20.9, 17.7, 15.5, 13.8, 13.3, 10.8.

### EXAMPLE 35

¹³C NMR (CDCl₃, 75 MHz) δ 216.5, 204.1, 203.7, 165.9, 165.6, 160.8, 157.2, 132.9, 129.9, 129.6, 127.6, 125.3, 120.9, 104.1, 98.9, 96.2, 91.9, 83.4, 80.2, 80.1, 78.7, 78.6, 77.0, 70.3, 69.6, 65.8, 58.1, 55.4, 51.1, 44.1, 40.5, 40.2, 38.4, 37.4, 28.2, 25.3, 25.0, 22.2, 21.1, 20.2, 17.6, 15.3, 13.7, 13.2, 10.6.

### EXAMPLE 36

¹³C NMR (75 MHz, CDCl₃) δ 216.9, 204.4, 203.8, 166.1, 165.8, 157.3, 151.9, 151.8, 143.3, 139.9, 123.7, 121.8, 103.8, 99.0, 96.3, 92.2, 83.6, 81.7, 80.4, 79.7, 78.6,70.1, 69.3, 65.9, 57.9, 50.9, 46.1, 44.2, 40.6, 40.3, 38.3, 37.4, 37.0, 25.3, 25.0, 22.2, 21.0, 20.3, 17.6, 15.3, 13.6, 13.2, 10.6.

### EXAMPLE 37

¹³C NMR (75 MHz, CDCl₃) δ 216.62, 204.1, 203.7, 165.9, 165.6, 161.2, 157.2, 133.0, 130.7, 129.2, 128.1, 125.84, 115.3, 104.1, 98.9, 96.2, 92.2, 83.5, 80.2, 79.9, 78.6, 78.5, 78.4, 73.9, 70.3, 69.7, 65.8, 58.0, 51.1, 48.1, 44.1, 40.5, 40.2, 38.4, 37.4, 30.8, 28.1, 25.4, 25.0, 22.2, 21.1, 20.2, 18.1, 17.6, 15.3, 13.7, 13.2, 10.6.

### EXAMPLE 39

¹³C NMR (CDCl₃, 75 MHz) δ 216.0, 204.2, 203.8, 165.9, 165.6, 157.4, 150.6, 104.2, 98.8, 91.3, 83.2, 80.6, 79.1, 72.9, 70.2, 69.7, 65.7, 58.3, 50.4, 44.0, 40.2, 39.7, 38.0, 37.6, 30.8, 28.1, 25.5, 25.4, 22.3, 21.1, 20.3, 17.7, 15.5, 13.9, 13.5, 10.6.

### EXAMPLE 41

¹³C NMR (CDCl₃, 75 MHz) δ 216.9, 204.2, 203.8, 166.0, 165.8, 160.4, 157.3, 154.4, 148.4, 104.2, 95.2, 83.5, 80.4, 79.9, 78.6, 75.1, 70.3, 69.8, 65.8, 58.1, 51.2, 44.2, 40.6, 40.2, 39.8, 38.5, 37.5, 28.1, 25.3, 25.0, 22.2, 21.2, 20.2, 17.6, 15.3, 13.7, 13.2, 10.6.

### EXAMPLE 43

¹³C NMR (CDCl₃, 75 MHz) δ 217.1, 205.2, 169.6, 160.4, 157.8, 154.3, 148.6, 121.2, 103.1, 94.7, 83.5, 79.8, 77.3, 77.2, 75.2, 70.2, 58.1, 51.6, 51.0, 46.7, 44.8, 40.2, 39.8, 38.8, 37.4, 28.2, 22.4, 21.2, 19.6, 17.9, 14.7, 14.5, 13.6, 13.5, 10.5.

The foregoing is merely illustrative of the invention and is not intended to limit the same to the disclosed compounds and proceses. Variations and changes which are obvious to one skilled in the art are intended to be within the scope and nature of the invention as defined in the claims.

What is claimed is:

1. A compound, or a salt, prodrug, or salt of a prodrug thereof, having formula (I) 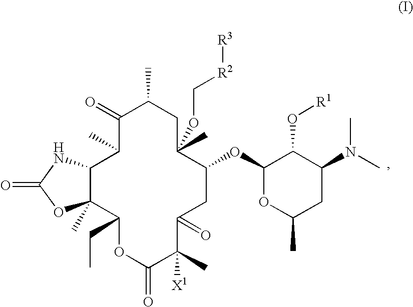
in which
R¹ is hydrogen or R^{p}, in which R^{p} is acetyl, benzoyl, trimethylsilyl, or triethylsilyl;
R² is —CH═CH— or —C≡—C—;
R³ is tetraazolyl or R⁴;
R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl, thienyl, 1,3,5-triazinyl, or 1,2,3-triazolyl; and
X¹ is hydrogen or fluoro;
in which each R⁴ moiety is connected through a carbon atom, substituted with one tetraazolyl substituent, and further unsubstituted or substituted with one or two substituents independently selected from the group consisting of alkyl, alkenyl, alkynyl, halo, —CN, —OH, —NH₂, —NH(alkyl), —N(alkyl)₂, —NO₂, —CF₃, —CH₂CF₃, —CF₂CF₃, —OCF₃, —OCH₂CF₃, —OCF₂CF₃, —C(O)H, —C(O)(alkyl), —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O) (alkyl), —NHC(O)O(alkyl), —NHC(O)NH₂, —NHC(O)NH(alkyl), and —NHC(O)N(alkyl)₂, and
in which the R³ tetraazolyl and the R⁴ tetraazolyl are connected through a carbon atom and are independently unsubstituted or substituted on one 1H or 2H nitrogen atom with one substituent selected from the group consisting of alkyl, —(CH₂)alkenyl, —(CH₂)alkynyl, and alkyl substituted with one substituent selected from the group consisting of halo, —CN, —OH, —NH₂, —NH(alkyl), —N(alkyl)₂, —CF₃, —CH₂CF₃, —CF₂CF₃, —C(O)H, ═O, —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂.

2. The compound of claim 1, or a salt, prodrug, or salt of a prodrug thereof, having formula (I), in which
R¹ is hydrogen;
R² is —CH═CH— or —C≡C—;
R³ is tetraazolyl or R⁴;
R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl, thienyl, 1,3,5-triazinyl, or 1,2,3-triazolyl; and
X¹ is hydrogen or fluoro;
in which each R⁴ moiety is connected through a carbon atom, substituted with one tetraazolyl substituent, and further unsubstituted or substituted with one or two substituents independently selected from the group consisting of alkyl, alkenyl, alkynyl, halo, —CN, —OH, —NH₂, —NH(alkyl), —N(alkyl)₂, —NO₂, —CF₃, —CH₂CF₃, —CF₂CF₃, —OCF₃, —OCH₂CF₃, —OCF₂CF₃, —C(O)H, —C(O)(alkyl), —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂, and
in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —CF₃, —CH₂CF₃, —CF₂CF₃, —C(O)H, ═O, —C(O)OH, —C(O)O(alkyl), —C(O)NH₂, —C(O)NH(alkyl), —C(O)N(alkyl)₂, —OC(O)(alkyl), —OC(O)O(alkyl), —OC(O)NH₂, —OC(O)NH(alkyl), —OC(O)N(alkyl)₂, —NHC(O)H, —NHC(O)(alkyl), —NHC(O)O(alkyl), —NHC(O)NH₂, —NHC(O)NH(alkyl), and —NHC(O)N(alkyl)₂.

3. The compound of claim 2, or a salt, prodrug, or salt of a prodrug thereof, having formula (I), in which
R¹ is hydrogen;
R² is —CH═CH— or —C≡C—;
R³ is tetraazolyl or R⁴;
R⁴ is furanyl, imidazolyl, isothiazolyl, isoxazolyl, naphthyl, 1,2,3-oxadiazolyl, oxazolyl, phenyl, pyrazinyl, pyrazolyl, pyridazinyl, pyrimidinyl, pyrrolyl, 1,3,4-thiadiazolyl, thiazolyl, pyridyl, thienyl, 1,3,5-triazinyl, or 1,2,3-triazolyl; and
X¹ is hydrogen or fluoro;
in which each R⁴ moiety is connected through a carbon atom, substituted with one tetraazolyl substituent, and further unsubstituted or substituted with one substituent selected from the group consisting of alkyl, halo, —CN, —OH, —NH₂, —NH(alkyl), —N(alkyl)₂, —NO₂, —CF₃, —C(O)H, —C(O)(alkyl), —C(O)OH, —C(O)O(alkyl), and —C(O)NH₂, and
in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —C(O)H, —C(O)OH, —C(O)O(alkyl), —C(O)NH₂, and —C(O)NH(alkyl).

4. The compound of claim 3, or a salt, prodrug, or salt of a prodrug thereof, having formula (I), in which
R¹ is hydrogen;
R² is —CH═CH— or —C≡C—;
R³ is tetraazolyl or R⁴;
R⁴ is thiazolyl, pyridyl, or thienyl; and
X¹ is hydrogen or fluoro;
in which each R⁴ moiety is connected through a carbon atom, substituted with one tetraazoiyl substituent, and further unsubstituted or substituted with one substituent selected from the group consisting of alkyl, halo, —CN, —OH, —NH₂, —NH(alkyl), —N(alkyl)₂, —NO₂, —CF₃, —C(O)H, —C(O)(alkyl), —C(O)OH, —C(O)O(alkyl), and —C(O)NH₂, and
in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —C(O)H, —C(O)OH, —C(O)O(alkyl), —C(O)NH₂, and —C(O)NH(alkyl).

5. The compound of claim 4, or a salt, prodrug, or salt of a prodrug thereof, having formula (I), in which
R¹ is hydrogen;
R² is —CH═CH— or —C≡C—;
R³ is tetraazolyl or R⁴;
R⁴ is thiazolyl, pyridyl, or thienyl; and
X¹ is hydrogen or fluoro;
in which each R⁴ moiety is connected through a carbon atom, substituted with one tetraazolyl substituent, and
in which the R³ tetraazolyl and the R⁴ tetraazolyl are connected through a carbon atom and are independently unsubstituted or substituted on a 1H or 2H nitrogen atom with one substituent selected from the group consisting of alkyl, —(CH₂)alkenyl, —(CH₂)alkynyl, and alkyl substituted with a substituent selected from the group consisting of halo, —CN, —OH, —NH₂, —NH(alkyl), —N(alkyl)₂, —C(O)H, —C(O)OH, —C(O)O(alkyl), —C(O)NH₂, and —C(O)NH(alkyl).

6. The compound of claim 5, or a salt, prodrug, or salt of a prodrug thereof, having formula (I), in which
R¹ is hydrogen;
R² is —CH═CH— or —C≡C—;
R³ is 1H-tetraazol-5-yl, 2H-tetraazol-5-yl, 1-methyl-1H-tetraazol-5-yl, 2-methyl-2H-tetraazol-5-yl, 2-allyl-2H-tetraazol-5-yl, 2-prop-2-ynyl-2H-tetraazol-5-yl, 2-(((methoxy)carbonyl)methyl)-2H-tetraazol-5-yl, 2-(2-(cyano)ethyl)-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and
X¹ is hydrogen or fluoro.

7. The compound of claim 1, or a salt, prodrug, or salt of a prodrug thereof, having formula (I) 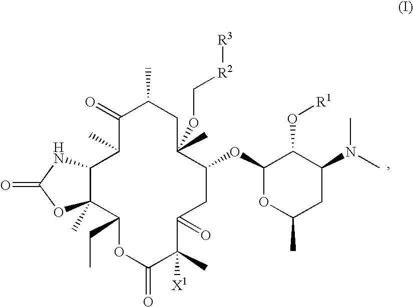
in which R¹ is hydrogen; R² is —CH═CH— or —C≡C—; R³ is tetraazolyl or R⁴; R⁴ is thiazolyl, pyridyl, or thienyl; and X¹ is hydrogen or fluoro;
in which each R⁴ moiety is substituted with one tetraazolyl substituent, the R³ tetraazolyl is substituted at the 1H or 2H nitrogen atom with alkyl, and the R⁴ tetraazolyl is unsubstituted or substituted at the 1H or 2H nitrogen atom with one substituent selected from the group consisting of alkyl, —(CH₂)alkenyl, and alkyl substituted with one substituent selected from the group consisting of —CN and —C(O)O(alkyl).

8. The compound of claim 1, or a salt, prodrug, or salt of a prodrug thereof, having formula (I) 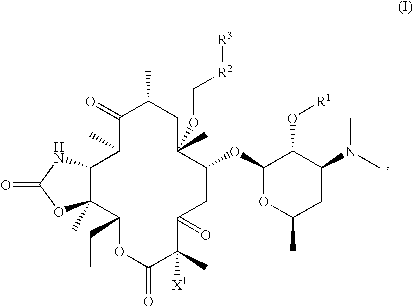
in which R¹ is hydrogen; R² is —CH═CH— or —C≡C—; R³ is tetraazolyl or R⁴; R⁴ is thiazolyl, pyridyl, or thienyl; and X¹ is hydrogen or fluoro;
in which each R⁴ moiety is substituted with one tetraazolyl substituent, the R³ tetraazolyl is substituted at the 1H or 2H nitrogen atom with C₁-alkyl, and the R⁴ tetraazolyl is unsubstituted or substituted at the 1H or 2H nitrogen atom with one substituent selected from the group consisting of C₁-alkyl, —(CH₂) —C₂-alkenyl, and C₁-C₂-alkyl substituted with one substituent selected from the group consisting of —CN and —C(O)O(C₁-alkyl).

9. The compound of claim 8, or a salt, prodrug, or salt of a prodrug thereof, having formula (I)
in which R¹ is hydrogen; R² is —CH═CH—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and X¹ is hydrogen.

10. The compound of claim 8, or a salt, prodrug, or salt of a prodrug thereof, having formula (I)
in which R¹ is hydrogen; R² is —CH═CH—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and X¹ is fluoro.

11. The compound of claim 8, or a salt, prodrug, or salt of a prodrug thereof, having formula (I)
in which R¹ is hydrogen; R² is —C≡C—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and X¹ is hydrogen.

12. The compound of claim 8, or a salt, prodrug, or salt of a prodrug thereof, having formula (I)
in which R¹ is hydrogen; R² is —C≡C—; R³ is 2-methyl-2H-tetraazol-5-yl, 5-(2-allyl-2H-tetraazol-5-yl)thien-2-yl, 5-(2-(2-(cyano)ethyl)-2H-tetraazol-5-yl)thien-2-yl, 5-(2-(methoxycarbonylmethyl)-2H-tetraazol-5-yl)-thien-2-yl, 6-(2-methyl-2H-tetraazol-5-yl)pyridin-3-yl, 2-(2-methyl-2H-tetraazol-5-yl)-1,3-thiazol-5-yl, 5-(2-methyl-2H-tetraazol-5-yl)thien-2-yl, 5-(1-methyl-1H-tetraazol-5-yl)thien-2-yl, or 5-(2H-tetraazol-5-yl)thien-2-yl; and X¹ is fluoro.

13. A composition for prophylaxis or treatment of bacterial infections in a fish or a mammal, the compositions comprising a therapeutically effective amount of a compound of claim 1 and an excipient.

14. A method for prophylaxis or treatment of bacterial infections in a fish or a mammal comprising administering to the fish or the mammal a therapeutically effective amount of a compound of claim 1.

15. A compound of claim 1, or a salt, prodrug, or salt of a prodrug thereof, which is
(3aS,4R,7S,9R,10R,11S,13R,15R,15aR)-4-ethyl-7-fluoro-3a,7,9,11,13,15-hexamethyl-l-((3-(2-(2-methyl-2H-tetraazol-5-yl)-1,3-thiazol-5-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
(3aS,4R,7R,9R,10R,11S,13R,15R,15aR)-4-ethyl-3a,7,9,11,13,15-hexamethyl-1′-((3-(2-(2-methyl-2H-tetraazol-5-yl)-1,3-thiazol-5-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
(3aS,4R,7R,9R,10R,11S,13R,15R,15aR)-4-ethyl-3a,7,9,11,13,15-hexamethyl-11-(((2E)-3-(5-(2-methyl-2H-tetraazol-5-yl)thien-2-yl)prop-2-enyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
(3aS,4R,7R,9R,10R,11S,13R,15R,15aR)-4-ethyl-3a,7,9,11,-13,15-hexamethyl-11-(((2E)-3-(5-(1-methyl-1H-tetraazol-5-yl)thien-2-yl)prop-2-enyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
(3aS,4R,7R,9R,10R,11S,13R,15R,15aR)-4-ethyl-3a,7,9,11,13,15-hexamethyl-2,6,8,14-tetraoxo-11-(((2E)-3-(5-(2H-tetraazol-5-yl)thien-2-yl)prop-2-enyl)oxy)-tetradecahydro-2H-oxacyclotetradecino(4,3-d)(1,3)oxazol-10-yl 3,4,6-trideoxy-3-(dimethylamino)-β-D-xylo-hexopyranoside;
(3aS,4R,7S,9R,10R,11S,13R,15R,15aR)-4-ethyl-7-fluoro-3a,7,9,11,13,15-hexamethyl-11-((3-(5-(2-methyl-2H-tetraazol-5-yl)thien-2-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
(3aS,4R,7R,9R,10R,11S,13R,15R,15aR)-4-ethyl-3a,7,9,11,13,15-hexamethyl-11-((3-(5-(2-methyl-2H-tetraazol-5-yl)thien-2-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
(3aS,4R,7S,9R,10R,11S,13R,15R,15aR)-4-ethyl-7-fluoro-3a,7,9,11,13,15-hexamethyl-11-(((2E)-3-(5-(2-methyl-2H-tetraazol-5-yl)thien-2-yl)prop-2-enyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
methyl (5-(5-(3-(((3aS,4R,7S,9R,10R,11S,13R,15R,15aR)-4-ethyl-7-fluoro-3a,7,9,11,13,15-hexamethyl-2,6,8,14-tetraoxo-10-((3,4,6-trideoxy-3-(dimethylamino)-beta-D-xylo-hexopyranosyl)oxy)tetradecahydro-2H-oxacyclotetradecino (4,3-d)(1,3)oxazol-11-yl)oxy)prop-1-ynyl)thien-2-yl)-2H-tetraazol-2-yl)acetate;
(3aS,4R,7S,9R,1R,11S,13R,15R,15aR)-4-ethyl-7-fluoro-3a,7,9,11,13,15-hexamethyl-11-((3-(6-(2-methyl-2H-tetraazol-5-yl)pyridin-3-yl)prop-2-ynyl)oxy)-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
(3aS,4R,7S,9R,10R,11S,13R,15R,15aR)-11-((3-(5-(2-allyl-2H-tetraazol-5-yl)thien-2-yl)prop-2-ynyl)oxy)-4-ethyl-7-fluoro-3a,7,9,11,13,15-hexamethyl-2,6,8,14-tetraoxotetradecahydro-2H-oxacyclotetradecino(4,3-d)(1,3)-oxazol-10-yl 3,4,6-trideoxy-3-(dimethylamino)-β-D-xylo-hexopyranoside;
3-(5-(5-(3-(((3aS,4R,7S,9R,10R,11S,13R,15R,15aR)-4-ethyl-7-fluoro-3a,7,9,11,13,15-hexamethyl-2,6,8,14-tetraoxo-10-((3,4,6-trideoxy-3-(dimethylamino)-β-D-xylo-hexopyranosyl)oxy)tetradecahydro-2H-oxacyclotetradecino-(4,3-d)(1,3)oxazol-11-yl)oxy)prop-1-ynyl)thien-2-yl)-2H-tetraazol-2-yl)propanenitrile; or
(3aS,4R,7S,9R,10R,11S,13R,15R,15aR)-4-ethyl-7-fluoro-3a,7,9,11,13,15-hexamethyl-11-((3-(2-methyl-2H-tetraazol-5-yl)prop-2-ynyl)oxy)-2,6,8,14-tetraoxotetradecahydro-2H-oxacyclotetradecino(4,3-d)(1,3)oxazol-10-yl 3,4,6-trideoxy-3-(dimethylamino)-β-D-xylo-hexopyranoside.
